(19) United States
(12) Patent Application Publication
(10) Pub. No.: US 2014/0030193 A1
(43) Pub. Date: Jan. 30, 2014

(54) CUINSE/ZNS NIR-QUANTUM DOTS (QDS) FOR BIOMEDICAL IMAGIING

(71) Applicants: Peter C. Searson, Baltimore, MD (US); Justin Galloway, Odenton, MD (US); Kwan Hyi Lee, Seoul (KR); Jea Ho Park, Baltimore, MD (US)

(72) Inventors: Peter C. Searson, Baltimore, MD (US); Justin Galloway, Odenton, MD (US); Kwan Hyi Lee, Seoul (KR); Jea Ho Park, Baltimore, MD (US)

(73) Assignee: The Johns Hopkins University, Baltimore, MD (US)

(21) Appl. No.: 14/111,158

(22) PCT Filed: Apr. 11, 2012
(86) PCT No.: PCT/US12/33094

§ 371 Date: Oct. 10, 2013

Related U.S. Application Data
(60) Provisional application No. 61/474,037, filed on Apr. 11, 2011.

(51) Int. Cl. A61K 49/00

(52) U.S. Cl. CPC A61K 49/0067; A61K 49/0058
USPC 424/9.6

(57) ABSTRACT

Applications in nanomedicine, such as diagnostics and targeted therapeutics, rely on the detection and targeting of membrane biomarkers. Disclosed herein are functionalized quantum dots exhibiting greater stability in water, methods of making the functionalized quantum dots and methods of in vivo imaging using the functionalized quantum dots.

### CROSS-REFERENCE TO RELATED APPLICATIONS

This application claims benefit of priority to provisional patent application No. 61/474,037, filed Apr. 11, 2011, the entire contents of which are incorporated by reference.

### STATEMENT REGARDING FEDERALLY SPONSORED RESEARCH OR DEVELOPMENT

This invention was made with U.S. Government support of Grant No. U54CA151838 awarded by the Center for Cancer Nanotechnology Excellence. The U.S. Government has certain rights in this invention.

### BACKGROUND OF THE INVENTION

The detection of cancer biomarkers is important for diagnosis, disease stage forecasting, and clinical management. Since tumor populations are inherently heterogeneous, a key challenge is the quantitative profiling of membrane biomarkers, rather than secreted biomarkers, at the single cell level. The detection of cancer biomarkers is also important for imaging and therapeutics since membrane proteins are commonly selected as targets. Many methods for detection of membrane proteins yield ensemble averages and hence have limited application for analysis of heterogeneous populations or single cells. Fluorescence-based methods allow detection at the single cell level, however, photobleaching presents a major limitation in obtaining quantitative information. Quantum dots overcome the limitations associated with photobleaching, however, realizing quantitative profiling requires stable quantum yield, monodisperse quantum dot-antibody (QD-Ab) conjugates, and well-defined surface chemistry. (Resch-Genger, U.; Grabolle, M.; Cavaliere-Jaricot, S.; Nitschke, R.; Nann, T., Quantum dots versus organic dyes as fluorescent labels. Nature Methods 2008, 5, (9), 763-775.) By quantitative profiling we specifically refer to methods that yield absolute values of expression levels (e.g. #μm⁻²) and not relative values.

Pancreatic cancer is the fourth leading cause of cancer death in the US (about 35,000 per year). (American Cancer Society, A. Cancer Facts and figures 2009; American Cancer Society: Atlanta, Ga., 2009.) The survival rate amongst pancreatic cancer patients is extremely low, primarily due to the fact that a large fraction (about 80%) of tumors is metastatic at the time of diagnosis. (Hezel, A. F.; Kimmelman, A. C.; Stanger, B. Z.; Bardeesy, N.; Depinho, R. A., Genetics and biology of pancreatic ductal adenocarcinoma. Genes and Development 2006, 20, (10), 1218-1249.) The histologic progression from non-invasive precursor lesions, pancreatic intraepithelial neoplasia (PanINs), to invasive and metastatic pancreatic cancer is associated with the sequential accumulation of molecular alterations. (Maitra, A.; Hruban, R. H., Pancreatic cancer. Annu Rev Pathol 2008, 3, 157-188.) (Harsha, H. C.; Kandasamy, K.; Ranganathan, P.; Rani, S.; Ramabadran, S.; Gollapudi, S.; Balakrishnan, L.; Dwivedi, S. B.; Telikicherla, D.; Selvan, L. D. N.; Goel, R.; Mathivanan, S.; Marimuthu, A.; Kashyap, M.; Vizza, R. F.; Mayer, R. J.; DeCaprio, J. A.; Srivastava, S.; Hanash, S. M.; Hruban, R. H.; Pandey, A., A Compendium of Potential Biomarkers of Pancreatic Cancer. Plos Medicine 2009, 6, (4), E1000046.)

A current challenge in biomedical imaging is the synthesis of water soluble QDs with emission wavelength in the near-IR, high quantum yield, stability in water, and relatively small size. Ideally the synthesis should be relatively straightforward and not involve toxic elements. The optimum wavelength for in vivo optical imaging, taking into account the absorbance from melanin in the epidermis, hemoglobin in blood, and water in tissue, is in the range from 700-900 nm. (W. F. Cheong, S. A. Prahl, A. J. Welch, IEEE Journal of Quantum Electronics 1990, 26, 2166.) (K. Konig, Journal of Microscopy 2000, 200, 83.) To achieve emission in this optical window, requires a QD with a band gap of around 1.3-1.7 eV. (I. L. Medintz, H. T. Uyeda, E. R. Goldman, H. Mattoussi, Nat Mater 2005, 4, 435.) Semiconductor QDs that emit in the near-IR, such as CdTe, PbS, InAs, InP, have been synthesized (B. Blackman, D. Battaglia, X. G. Peng, Chem Mater 2008, 20, 4847.) (D. Battaglia, X. G. Peng, Nano Letters 2002, 2, 1027.) (J. Y. Chang, S. R. Wang, C. H. Yang, Nanotechnology 2007, 18.) (M. A. Hines, G. D. Scholes, Adv Mater 2003, 15, 1844.) (W. Lin, K. Fritz, G. Guerin, G. R. Bardajee, S. Hinds, V. Sukhovatkin, E. H. Sargent, G. D. Scholes, M. A. Winnik, Langmuir 2008, 24, 8215.) and explored for biomedical imaging (J. P. Zimmer, S. W. Kim, S. Ohnishi, E. Tanaka, J. V. Frangioni, M. G. Bawendi, Journal of the American Chemical Society 2006, 128, 2526.) (R. G. Xie, X. G. Peng, Angewandte Chemie-International Edition 2008, 47, 7677.) (S. Kim, Y. T. Lim, E. G. Soltesz, A. M. De Grand, J. Lee, A. Nakayama, J. A. Parker, T. Mihaljevic, R. G. Laurence, D. M. Dor, L. H. Cohn, M. G. Bawendi, J. V. Frangioni, Nature Biotechnology 2004, 22, 93.) (R. Hu, K. T. Yong, I. Roy, H. Ding, W. C. Law, H. X. Cai, X. H. Zhang, L. A. Vathy, E. J. Bergey, P. N. Prasad, Nanotechnology 2010, 21.).

High quantum yield is important to optimize the signal-to-noise ratio for imaging, and stability in aqueous solutions is key to avoid aggregation and degradation during imaging. At the same time, it is thought that a hydrodynamic diameter less than about 15 nm is necessary to ensure renal clearance and to avoid accumulation in other organs. (H. S. Choi, W. Liu, P. Misra, E. Tanaka, J. P. Zimmer, B. I. Ipe, M. G. Bawendi, J. V. Frangioni, Nature Biotechnology 2007, 25, 1165.) In addition, due to concerns over toxicity if QDs are not cleared from the body, it is desirable to avoid elements such as cadmium, lead, and arsenic. Thus there remains a need for the development of QD systems that satisfy all of these requirements.

### BRIEF SUMMARY OF THE INVENTION

Disclosed herein is a one-pot synthesis of CuInₓSe_{y}/ZnS core/shell QDs with an emission wavelength λ>700 nm. The 20% quantum yield of the core increases to as high as 60% after passivation with ZnS. After thiolation and lipid coating, the CuInₓSe_{y}/ZnS/DDT/lipid QDs are stable in water for about a week and maintain high quantum yield. Also disclosed is in vivo fluorescence imaging in a mouse model, illustrating uniform intensity that can be resolved without any image processing.

The CuInₓSe_{y} QDs were synthesized by reaction of CuI, InI₃, and bis(trimethylsilyl) selenide ((TMS)₂Se) in trioctylphosphine oxide (TOPO) and hexadecylamine (HDA). The Cu:In:Se precursor ratio was 1:4:14, in order to achieve the desired end ratio where x ranges between 1-4 and y ranges between 2-6. The composition of the QDs will vary based on temperature or concentration of the reactants. After injection of the precursors at 270° C. for 6 s, the reaction was quenched by injection of hexane. Large CuInSe₂ nanoparticles have been synthesized from Cu, In, and Se precursors in oleyamine (OA), (M. E. Norako, R. L. Brutchey, Chem Mater 2010, 22, 1613.) (Q. Guo, S. J. Kim, M. Kar, W. N. Shafarman, R. W. Birkmire, E. A. Stach, R. Agrawal, H. W. Hillhouse, Nano Letters 2008, 8, 2982.) and CuInₓSe_{y} QDs have been synthesized from TOPO and OA. (P. M. Allen, M. G. Bawendi, Journal of the American Chemical Society 2008, 130, 9240.) (E. Cassette, T. Pons, C. Bouet, M. Helle, L. Bezdetnaya, F. Marchal, B. Dubertret, Chem Mater 2010, 22, 6117.) However, we were not able to grow an effective passivation layer on CuInₓSe_{y} cores synthesized in these solvents. After numerous investigations using varying combinations of the solvents TOPO, trictylphosphine (TOP), HDA, and OA, we unexpectedly discovered that the synthesis of CuInₓSe_{y} cores in TOPO and HDA with a mole ratio of about 1:3 was optimum to produce QDs producing high quantum yield, and improved stability in water.

### BRIEF DESCRIPTION OF THE DRAWINGS

FIGS. 1-6 relate to Example 1

FIG. 1. (a) Schematic illustration of QD conjugates for biomarker targeting: (QD-L-PEG) CdSe/(Cd,Zn)S QDs with 80 mol % MHPC and 20 mol % DPE-Peg 2k. (QD-L-COOH) QDs with 80 mol % MHPC, 15 mol % DPE-PEG2k, and 5 mol % DPE-PEG2k-COOH. (QD-L-Ab) QD-L-COOH covalently conjugated with an average of three targeting antibodies per QD. (b) Particle size distributions for QD conjugates. (c) Zeta potential for QD conjugates. A zeta potential of about −10 mV minimizes aggregation and non-specific binding. (d) Absorbance and emission spectra for QD-L-PEG (Em. 623 nm) in water. (e) Quantum yield for QD conjugates in water.

FIG. 2. Profiling of biomarkers for pancreatic cancer. Fluorescence images of pancreatic cancer cells (Panc-1, MIA Paca-2, and Capan-1) and normal pancreatic cells (HPDE) incubated with 20 pmol QD-Ab conjugates (Ab=aPSCA, aCLDN4, and aMSLN).

FIG. 3. Quantitative analysis of pancreatic cancer biomarkers from fluorescence images with QD-Ab conjugates. (a) Saturation of membrane biomarkers. Average fluorescence intensity for Panc-1 cells incubated with different concentrations of QD-aMSLN. The error bars represent the standard error for measurements over at least 30 cells. The slope at lower concentrations is 1.0 confirming negligible non-specific binding or competitive binding. The plateau at 10 mmol QDs indicates saturation of MSLN at the surface. (b) Stability of fluorescence in QDs and fluorophores. Average fluorescence intensity for Panc-1 cells incubated with QD-aCLDN4 conjugates or PE (phycoerythrin)-aCLDN4 conjugates versus illumination time. (c) Calibration of QD fluorescence. Fluorescence images for different concentrations of QDs confined between two glass slides with fixed area. Top row: 36, 360, 1087 QDs μm⁻², bottom row: 1813, 2513, 2900 QDs μm⁻². (d) Average fluorescence intensity (normalized for 0.5 s exposure time) versus QD concentration obtained from analysis of images of QD suspensions.

FIG. 4. Absolute expression levels for biomarkers for pancreatic cancer. Average biomarker density per μm² for PSCA, claudin-4 and mesothelin in the three pancreatic cancer cell lines obtained from the average fluorescence intensity per cell and the calibration curve. Data were obtained from at least 300 Capan-1 cells, 100 MIAPaCa-2 cells, and 50 Panc-1 cells. Error bars represent the standard error.

FIG. 5. Spatial distribution of biomarkers. (a) Spatial distribution of mesothelin expression levels over a Panc-1 cell (inset). (b) Quantitative linear profiling of the claudin-4 density across a capan-1 cell (inset). The profiles were along radial lines separated by 22.5° and normalized to the cell diameter.

FIG. 6. Multiplexed imaging of cancer biomarkers on MIAPaCa-2 cells. Absorbance and emission spectra for (a) QD(Em.524)-L-aCLDN4, (b) QD(Em.623)-L-aMSLN, and (c) QD(Em.707)-L-aPSCA. (d) Phase contrast microscope image for MIAPaCa-2 cells after incubation with the three QD-Ab conjugates. Fluorescence images obtained with (e) FTIC (517/40, green), (f) TRITC (605/40, red), and (g) NIR (665 LP, infra red) filters. (h) Average biomarker density per cell for PSCA, claudin-4 and mesothelin in MIAPaCa-2 cells measured simultaneously. Standard error obtained from 150 cells.

FIG. 7. (a) Photoluminescence spectra for CuInₓSe_{y} (745 nm peak and 133 nm FWHM) and CuInₓSe_{y}/ZnS QDs (737 nm peak with 175 nm FWHM), and absorbance spectrum for CuInₓSe_{y}/ZnS QDs. Inset shows a photograph of suspensions of CuInₓSe_{y} (left) and CuInₓSe_{y}/ZnS (right) QDs in chloroform under UV excitation. The quantum yield increased from 20% to 50% after ZnS passivation. (b) EDS spectrum QDs and high resolution TEM image for a CuInSe QD. (c) EDS spectrum and high resolution TEM image for a CuInₓSe_{y}/ZnS QD. The gold peaks in the spectra are from the TEM grid. The average diameter, obtained from analysis of TEM images, is 4.0±0.13 nm for the CuInₓSe_{y} cores and 5.0±0.17 nm for the CuInₓSe_{y}/ZnS core/shell QDs. (d) X-ray diffraction patterns for CuInₓSe_{y} and CuInₓSe_{y}/ZnS QDs. The peak positions for stannite form of CuIn₃Se₅ and their relative intensities are also shown.

FIG. 7-13 relate to Example 2

FIG. 8. (a) Quantum yield versus time for CuInₓSe_{y}, CuInₓSe_{y}/ZnS, and CuInₓSe_{y}/ZnS/DDT QDs in chloroform. (b) Quantum yield versus time for CuInₓSe_{y}/ZnS/DDT/lipid QDs in water. (c) Size distribution of CuInₓSe_{y}/ZnS/DDT/lipid QDs in water measured by DLS. The average diameter is 15 nm. The inset shows a schematic illustration of the functionalized QDs.

FIG. 9. Fluorescence images obtained from the ventral side of a mouse after tail vein injection of 230 pmol QDs. (a) Before tail vein injection, (b) 5 minutes post-injection, (c) 90 minutes post-injection, and (d) 48 hours post-injection. (e) Normalized average intensity per pixel (obtained from the fluorescence images) versus time after injection.

FIG. 10. (a) High resolution TEM image of several CuInSe QDs. (b) Same image with QDs indicated by circles.

FIG. 11. Low magnification TEM images of CuInSe/ZnS core/shell QDs.

FIG. 12. Size distribution for lipid coated CuInₓSe_{y}/ZnS QDs. From analysis of TEM images, the QDs are 5 nm in diameter. Taking the DDT inner leaflet as 1 nm, the lipid outer leaflet as 2 nm, and the PEG radius of gyration as 2 nm, we expect the lipid-coated QDs to have a diameter of 15 nm, in excellent agreement with the average obtained from the number density. The relatively small differences between the volume, number, and intensity distributions indicate a very small amount of aggregation.

FIG. 13. Average fluorescence intensity per pixel for different organs versus time after injection of lipid coated CuInₓSe_{y}/ZnS QDs. Organs were resected and imaged using the Li-cor imaging system. Each point is the average obtained from 5-6 mice, except for 48 h (3 mice).

### DETAILED DESCRIPTION OF THE INVENTION

In an embodiment, the invention relates to quantum dots with a CuInₓSe_{y}/ZnS core/shell, having an emission wavelength λ>700 nm, and an improved stability in water. In an embodiment, the invention relates to a method of preparing quantum dots that have an improved stability in water. In an embodiment, the invention relates to a method of preparing quantum dots synthesized by reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio. In an embodiment, the invention relates to quantum dots with a CuInₓSe_{y}/ZnS core/shell synthesized by reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio. In an embodiment, the invention relates to a method of in vivo imaging using quantum dots with a CuInₓSe_{y}/ZnS core/shell synthesized by reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio.

An improved stability in water includes stability of the QD in water, at room temperature for a period of one week or more. An improved stability in water or aqueous solution includes stability of the QD by itself or conjugated to an antibody, at room temperature for a period of one week or more.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

The lipid encapsulation of the QDs can be accomplished by formation of a lipid monolayer, similar to the outer leaflet of a bilayer membrane. A combination of single and double acyl chain phospholipids can be used to form the outer leaflet. Among non-limiting examples are the single alkyl chain phospholipid 1-myristoyl-2-hydroxy-sn-glycero-3-phosphocholine (MHPC) and the double alkyl chain lipid 1,2-distearoyl-sn-glycero-3-phophoethanolamine (DSPE). The DSPE may also be a pegylated DSPE (DSPE-PEG2k), which results in QD-L-PEG conjugates that are stable for several weeks. The quantity of the phospholipids can be as low as 20 mol % and as high as 80 mol % such that the ratios are 20 ml %:80 mol % up to and including 80 mo %:20 mol %. The quantity can include values at 20 mol %, 30 mol %, 40 mol %, 50 mol %, 60 mol %, 70 mol % and 80 mol %.

The QDs may undergo thiolation which involves dodecanethiol (DDT) functionalization. Thiolation increases the QD stability. Other thiolating agents are well known in the art and may be substituted in this process step.

The core of CuInₓSe_{y} can have an x value of 1-4 and a y value of 2-6. The composition of the resultant QDs will vary based on temperature or concentration of the reactants. It is well within the skill of one of ordinary skill in the art to adjust the reaction to produce a stoichiometry for a product with a CuInₓSe_{y} core where x=1-4 and y=2-6.

In an embodiment, the invention relates to a method of preparing quantum dots synthesized by reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio. In an embodiment, the method further comprises a thiolation step. In an embodiment, the method further comprises lipid encapsulation. In an embodiment, the method further comprises antibody conjugation to the QD.

In an embodiment, the invention relates to quantum dots with a CuInₓSe_{y}/ZnS core/shell synthesized by reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio. In an embodiment, the QD may be thiolated. In an embodiment, the QD may be lipid encapsulated. In an embodiment, the QD may be conjugated to an antibody.

In an embodiment, the invention relates to a method of in vivo imaging using quantum dots with a CuInₓSe_{y}/ZnS core/shell synthesized by reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio. In an embodiment, the method further comprises a thiolation step. In an embodiment, the method further comprises lipid encapsulation. In an embodiment, the method further comprises antibody conjugation to the QD. The antibody conjugated quantum dot may be administered to the subject by oral or parenteral routes, or by other means well known in the art. The subject may include invertebrate and vertebrate species. The imaging may include in utero imaging, whole body imaging or organ specific imaging. Imaging may occur via fluorescence scanning. Images can be recorded using digital scanning techniques, which are well known in the art. The images may be continuously scanned, or may include captured images, such as a photograph or time-lapsed recordings, all of which are well known in the art.

For the quantitative profiling of cancer biomarkers, we have selected three biomarkers for pancreatic cancer for quantitative imaging: prostate stem cell antigen (PSCA), claudin-4 (CLDN4), and mesothelin (MSLN). PSCA and MSLN are gylcosylphosphatidyl inositol (GPI)-anchored proteins whereas CLDN4 is one of a large family of tight junction proteins. PSCA is overexpressed in adenocarcinomas and present in the majority of PanIN lesions beginning with early PanIN-1. (Maitra, A.; Adsay, N. V.; Argani, P.; Iacobuzio-Donahue, C.; De Marzo, A.; Cameron, J. L.; Yeo, C. J.; Hruban, R. H., Multicomponent analysis of the pancreatic adenocarcinoma progression model using a pancreatic intraepithelial neoplasia tissue microarray. Mod Pathol 2003, 16, (9), 902-12.) (Maitra, A.; Iacobuzio-Donahue, C.; Argani, P.; Wilentz, R. E.; Cameron, J. L.; Yeo, C. J.; Kern, S. E.; Goggins, M. G.; Hruban, R. H., Expression of mesothelin and prostate stem cell antigen, two novel markers identified by serial analysis of gene expression, in mucinous cystic neoplasms and intraductal papillary mucinous neoplasms of the pancreas. Modern Pathology 2002, 15, (1), 137A-137A.) (Wente, M. N.; Jain, A.; Kono, E.; Berberat, P. O.; Giese, T.; Reber, H. A.; Friess, H.; Buchler, M. W.; Reiter, R. E.; Hines, O. J., Prostate stem cell antigen is a putative target for immunotherapy in pancreatic cancer. Pancreas 2005, 31, (2), 119-125.) Claudin-4 overexpression is observed in intermediate PanIN-2 lesions. (Michl, P.; Buchholz, M.; Rolke, M.; Kunsch, S.; Lohr, M.; McClane, B.; Tsukita, S.; Leder, G.; Adler, G.; Gress, T. M., Claudin-4: A new target for pancreatic cancer treatment using Clostridium perfringens enterotoxin. Gastroenterology 2001, 121, (3), 678-684.) (Nichols, L. S.; Ashfaq, R.; Iacobuzio-Donahue, C. A., Claudin 4 protein expression in primary and metastatic pancreatic cancer—Support for use as a therapeutic target. American Journal of Clinical Pathology 2004, 121, (2), 226-230.) (Morin, P. J., Claudin proteins in human cancer: promising new targets for diagnosis and therapy. Cancer Res 2005, 65, (21), 9603-6.) Mesothelin overexpression is a late event in the progression model of pancreatic cancer, almost always associated with invasion. (Maitra, A.; Iacobuzio-Donahue, C.; Argani, P.; Wilentz, R. E.; Cameron, J. L.; Yeo, C. J.; Kern, S. E.; Goggins, M. G.; Hruban, R. H., Expression of mesothelin and prostate stem cell antigen, two novel markers identified by serial analysis of gene expression, in mucinous cystic neoplasms and intraductal papillary mucinous neoplasms of the pancreas. Modern Pathology 2002, 15, (1), 137A-137A.) (Li, M.; Bharadwaj, U.; Zhang, R. X.; Zhang, S.; Mu, H.; Fisher, W. E.; Brunicardi, F. C.; Chen, C. Y.; Yao, Q. Z., Mesothelin is a malignant factor and therapeutic vaccine target for pancreatic cancer. Molecular Cancer Therapeutics 2008, 7, (2), 286-296.) (Argani, P.; Iacobuzio-Donahue, C.; Ryu, B.; Rosty, C.; Goggins, M.; Wilentz, R. E.; Murugesan, S. R.; Leach, S. D.; Jaffee, E.; Yeo, C. J.; Cameron, J. L.; Kern, S. E.; Hruban, R. H., Mesothelin is overexpressed in the vast majority of ductal adenocarcinomas of the pancreas: Identification of a new pancreatic cancer marker by serial analysis of gene expression (SAGE). Clinical Cancer Research 2001, 7, (12), 3862-3868.) All three of these biomarkers are therapeutic targets for pancreatic cancer. Quantitative profiling of these biomarkers was studied in three pancreatic cancer cell lines: Panc-1 (derived from pancreatic ductal adenocarcinoma), MIA PaCa-2 (derived from epithelial pancreatic carcinoma cells), and Capan-1 (derived from a liver metastasis of a grade II pancreatic adenocarcinoma). The immortalized pancreatic ductal cell line HPDE was used for comparison.

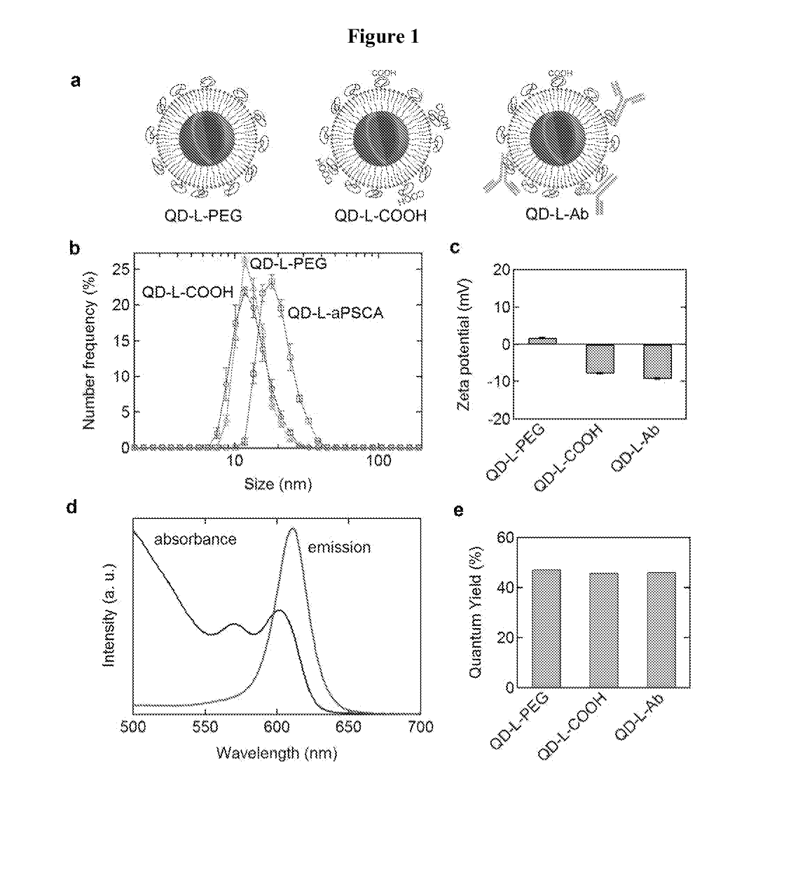

QDs exhibit size-dependent absorption and emission properties, (Brus, L. E., Electron Electron and Electron-Hole Interactions in Small Semiconductor Crystallites—the Size Dependence of the Lowest Excited Electronic State. Journal of Chemical Physics 1984, 80, (9), 4403-4409.) high fluorescence quantum yields, and with careful functionalization have been widely used for imaging and sensing. (Michalet, X.; Pinaud, F. F.; Bentolila, L. A.; Tsay, J. M.; Doose, S.; Li, J. J.; Sundaresan, G.; Wu, A. M.; Gambhir, S. S.; Weiss, S., Quantum dots for live cells, in vivo imaging, and diagnostics. Science 2005, 307, (5709), 538-544.) (Medintz, I. L.; Uyeda, H. T.; Goldman, E. R.; Mattoussi, H., Quantum dot bioconjugates for imaging, labelling and sensing. Nature Materials 2005, 4, (6), 435-446.) (Gao, X. H.; Cui, Y. Y.; Levenson, R. M.; Chung, L. W. K.; Nie, S. M., In vivo cancer targeting and imaging with semiconductor quantum dots. Nature Biotechnology 2004, 22, (8), 969-976.) (Sapsford, K. E.; Pons, T.; Medintz, I. L.; Mattoussi, H., Biosensing with luminescent semiconductor quantum dots. Sensors 2006, 6, (8), 925-953.) (Choi, H. S.; Liu, W.; Misra, P.; Tanaka, E.; Zimmer, J. P.; Itty Ipe, B.; Bawendi, M. G.; Frangioni, J. V., Renal clearance of quantum dots. Nat Biotechnol 2007, 25, (10), 1165-70.) (Gao, J.; Chen, K.; Xie, R.; Xie, J.; Yan, Y.; Cheng, Z.; Peng, X.; Chen, X., In vivo tumor-targeted fluorescence imaging using near-infrared non-cadmium quantum dots. Bioconjug Chem 2010, 21, (4), 604-9.) (Fu, A. H.; Gu, W. W.; Larabell, C.; Alivisatos, A. P., Semiconductor nanocrystals for biological imaging. Current Opinion in Neurobiology 2005, 15, (5), 568-575.) (Ballou, B.; Lagerholm, B. C.; Ernst, L. A.; Bruchez, M. P.; Waggoner, A. S., Noninvasive imaging of quantum dots in mice. Bioconjugate Chemistry 2004, 15, (1), 79-86.) (Smith, B. R.; Cheng, Z.; De, A.; Koh, A. L.; Sinclair, R.; Gambhir, S. S., Real-time intravital imaging of RGD-quantum dot binding to luminal endothelium in mouse tumor neovasculature. Nano Letters 2008, 8, (9), 2599-2606.) (Park, J.; Dvoracek, C.; Lee, K. H.; Galloway, J. F.; Bhang, H. E.; Pomper, M. G.; Searson, P. C., CuInSe/ZnS Core/Shell NIR Quantum Dots for Biomedical Imaging. Small 2011, 7, (22), 3148-52.) Quantitative QD-Ab targeting requires that each target molecule (e.g. membrane protein) is conjugated with one QD and that non-specific binding is minimized. Although various functionalization schemes have been reported in the literature, (Medintz, I. L.; Uyeda, H. T.; Goldman, E. R.; Mattoussi, H., Quantum dot bioconjugates for imaging, labelling and sensing. Nature Materials 2005, 4, (6), 435-446.) (Gao, X. H.; Cui, Y. Y.; Levenson, R. M.; Chung, L. W. K.; Nie, S. M., In vivo cancer targeting and imaging with semiconductor quantum dots. Nature Biotechnology 2004, 22, (8), 969-976.) (Dubertret, B.; Skourides, P.; Norris, D. J.; Noireaux, V.; Brivanlou, A. H.; Libchaber, A., In vivo imaging of quantum dots encapsulated in phospholipid micelles. Science 2002, 298, (5599), 1759-1762.) (Liu, J.; Lau, S. K.; Varma, V. A.; Moffitt, R. A.; Caldwell, M.; Liu, T.; Young, A. N.; Petros, J. A.; Osunkoya, A. O.; Krogstad, T.; Leyland-Jones, B.; Wang, M. D.; Nie, S. M., Molecular Mapping of Tumor Heterogeneity on Clinical Tissue Specimens with Multiplexed Quantum Dots. Acs Nano 2010, 4, (5), 2755-2765.) (Howarth, M.; Liu, W. H.; Puthenveetil, S.; Zheng, Y.; Marshall, L. F.; Schmidt, M. M.; Wittrup, K. D.; Bawendi, M. G.; Ting, A. Y., Monovalent, reduced-size quantum dots for imaging receptors on living cells. Nature Methods 2008, 5, (5), 397-399.) (Mulder, W. J. M.; Strijkers, G. J.; van Tilborg, G. A. F.; Cormode, D. P.; Fayad, Z. A.; Nicolay, K., Nanoparticulate Assemblies of Amphiphiles and Diagnostically Active Materials for Multimodality Imaging. Accounts of Chemical Research 2009, 42, (7), 904-914.) (Louie, A., Multimodality Imaging Probes: Design and Challenges. Chemical Reviews 2010, 110, (5), 3146-3195.) here we have developed a method based on encapsulation with a lipid layer (Dubertret, B.; Skourides, P.; Norris, D. J.; Noireaux, V.; Brivanlou, A. H.; Libchaber, A., In vivo imaging of quantum dots encapsulated in phospholipid micelles. Science 2002, 298, (5599), 1759-1762.) (Cormode, D. P.; Skajaa, T.; van Schooneveld, M. M.; Koole, R.; Jarzyna, P.; Lobatto, M. E.; Calcagno, C.; Barazza, A.; Gordon, R. E.; Zanzonico, P.; Fisher, E. A.; Fayad, Z. A.; Mulder, W. J. M., Nanocrystal Core High-Density Lipoproteins: A Multimodality Contrast Agent Platform. Nano Letters 2008, 8, (11), 3715-3723.) (Carion, 0.; Mahler, B.; Pons, T.; Dubertret, B., Synthesis, encapsulation, purification and coupling of single quantum dots in phospholipid micelles for their use in cellular and in vivo imaging. Nature Protocols 2007, 2, (10), 2383-2390.) (Koole, R.; van Schooneveld, M. M.; Hilhorst, J.; Castermans, K.; Cormode, D. P.; Strijkers, G. J.; Donega, C. D.; Vanmaekelbergh, D.; Griffioen, A. W.; Nicolay, K.; Fayad, Z. A.; Meijerink, A.; Mulder, W. J. M., Paramagnetic Lipid-Coated Silica Nanoparticles with a Fluorescent Quantum Dot Core: A New Contrast Agent Platform for Multimodality Imaging. Bioconjugate Chemistry 2008, 19, (12), 2471-2479.) optimized for quantitative targeting (FIG. 1a). Through a systematic study of functionalization parameters, the inventors disclose that unexpectedly: (1) functionalization of QDs can be achieved with commercially available reagents, (2) the yield of the functionalization process is high, (3) the QDs and QD-conjugates are monodisperse and exhibit improved stability in water, and (4) the functionalization method minimizes non-specific binding to cells.

### Example 1

### Profiling of Cancer Biomarkers

### Methods

### Synthesis of QDs

Most experiments were performed using CdSe/(Cd,Zn)S core/shell QDs with an emission wavelength of about 610 nm. (Park, J.; Lee, K. H.; Galloway, J. F.; Searson, P. C., Synthesis of Cadmium Selenide Quantum Dots from a Non-Coordinating Solvent: Growth Kinetics and Particle Size Distribution. Journal of Physical Chemistry C 2008, 112, (46), 17849-17854.) (Galloway, J. F.; Park, J.; Lee, K. H.; Wirtz, D.; Searson, P. C., Exploiting Nucleation and Growth in the Synthesis and Electrical Passivation of CdSe Quantum Dots. Science of Advanced Materials 2009, 1, (1), 1-8.) For multiplexing experiments we synthesized CdSe/(Cd,Zn)S core/shell QDs with an emission wavelength of 524 nm and CuInSe/ZnS core/shell QDs with an emission wavelength of 707 nm.

### Water Solubilization of QDs

Water soluble QDs were obtained by forming a lipid monolayer composed of MHPC/DPPE-PEG2k (80:20 mol %) or MHPC/DPPE-PEG2k/DPPE-PEG2k-COOH (80:15:5 mole %). Typically 0.25 nmol of QDs, 4 μmol of MHPC, 0.75 μmol of DPPE-PEG2k, and 0.25 μmol of DPPE-PEG2k-COOH were dissolved in 0.3 mL of chloroform. This solution was added to 2 ml of deionized water and heated and maintained at 110° C. for 1 h under vigorous stirring to evaporate chloroform. The resulting solution was sonicated for 1 h, centrifuged, and the supernatant then passed through a syringe filter with a 200 nm PTFE membrane (VWR) to remove any aggregates or unsuspended QDs. Quantum yield measurements were performed on suspensions with about 100 pmol QDs in 4 mL DI water using a Hamamatsu C9920-02 fluorometer.

### Cell Lines

A panel of three human pancreatic cancer cell lines (MIAPaCa-2, Panc-1, and Capan-1) were utilized for these studies. Mia PaCa-2 and Panc-1 were cultured with a growth medium containing DMEM (Dulbecco's Modified Eagle's Medium) as the base medium, FBS (fetal bovine serum, 10%), and P/S (penicillin/streptomycin, 1%), and Capan-1 was cultured in IMDM (Iscove's Modified Dulbecco's Medium) supplemented with 20% FBS and 1% P/S. All three cell lines were incubated at 37° C. and in 5% CO₂. The immortalized normal pancreatic cell line HPDE (human pancreatic duct epithelium) was used as a control. HPDE cells were cultured in keratinocyte serum-free (KSF) medium supplemented by bovine pituitary extract and epidermal growth factor (Gibco-BRL, Grand Island, N.Y.).

### Antibodies and Antibody Conjugation

QDs were conjugated with one of three antibodies: anti-Prostate Stem Cell Antigen (aPSCA), anti-claudin-4 (aCLDN4), or anti-mesothelin (aMSLN). The reaction of the primary amines on the antibody with lipid-modified QDs (carboxylic acid-terminated QDs) is catalyzed by 1-ethyl-3[3-dimethylaminopropyl]carbodiimide hydrochloride (EDC) resulting in the formation of an amide bond. In a typical reaction, 1 μM QDs was mixed with 2 mM EDC and 5 mM sulfo-NHS in 0.1 M MES (pH 6.0) and incubated for 15 minutes at room temperature with gentle mixing. The remaining unreacted EDC was quenched with the addition of 20 μL of 2-mercaptoethanol (1 M) for 10 minutes. Unreacted reagents and byproducts were removed by centrifugation in 100 kDa MWCO microcentrifuge tubes at 1000 g for 5 minutes. The activated QDs were then resuspended in 1×PBS. The activated QD stock solution was mixed with antibody solution (0.5-1 mg mL⁻¹ in PBS) to obtain a 3-6 fold molar excess of the antibodies to QDs. The reaction solution was incubated at room temperature for 2 h with gentle mixing. For control experiments QDs were prepared by coating with 80 mol % MHPC and 20 mol % PEGylated lipid DPE-PEG2k (no Ab). To remove excess reagents microfiltration was performed. To ensure that any aggregates are removed, an additional filtration step was carried out using syringe type filters (pore size: 100 nm). The QD suspensions were then characterized using UV-Vis absorption, photoluminescence (PL), dynamic light scattering (DLS), and surface charge (zeta potential).

### Imaging

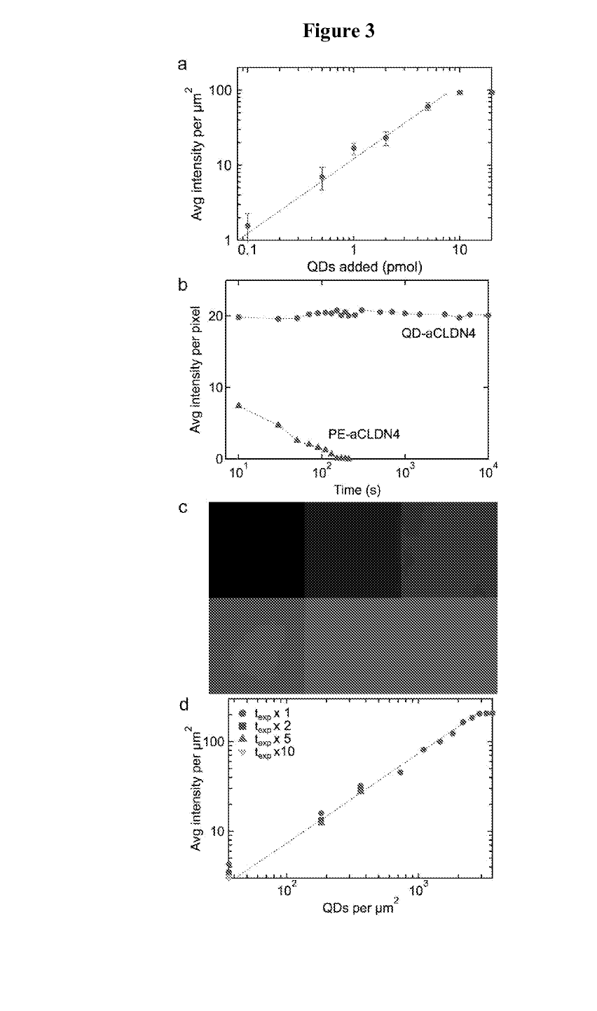

Briefly, about 10⁵ cells (see above for description of cell lines) were pre-seeded in a 12-well culture dish. At 50-70% confluency (1-2 days), the cell medium was aspirated and the cells washed three times with PBS. Fixing solution (3.7% Formaldehyde) was added to the wells for 20 min and washed three times with PBS. The cells were then incubated with a blocking buffer (10% horse serum or 5% BSA in PBS) for 1 h prior to introducing 500 μL of QD-Ab conjugates to each well and then incubated at RT for 30 min. In all profiling experiments, cells were incubated with 20 pmol QDs corresponding to a dose of about 10⁸ QDs per cell. In experiments to confirm that the membrane biomarkers were saturated with QD-Ab conjugates (FIG. 3a), cells were incubated with 0.1-20 pmol QDs. Next, the QD-Ab solution was aspirated and the cells washed with PBS three times. The maximum biomarker density (around 500 μm⁻²) corresponds to about 10⁵ per cell or a maximum QD excess of about 1000 QDs per biomarker.

Phase contrast and fluorescence images were taken with a Nikon ECLIPSE TE2000-U microscope equipped with a filter wheel allowing us to mix-and-match excitation and emission filters depending on the QDs (Ex: 350/50, 484/15, 555/25; Em: 457/30, 517/40 (FITC), 605/40 (TRITC), 620/40, or 665/LP). For experiments with QDs (Em. 607 nm), we used Ex: 555/25 and Em: 605/40. All images were obtained with a ×20 objective using Nikon Elements software. The focus was set to the top surface of the cell rather than the bottom surface of the cell on the glass slide. Images were recorded using a CoolSNAP HQ² camera with 2×2 binning yielding 696×520 pixels, and an output intensity range from 0-255. The exposure time was 0.5 s unless otherwise indicated.

### Flow Cytometry Analysis

Cell were centrifuged at 500×g for 5 mins and washed three times in an isotonic PBS buffer supplemented with 0.5% BSA to remove contaminating serum components that may be presented in the culture medium. Cells were resuspended in the same buffer to a final concentration of 4×10⁶ cells mL⁻¹ and 25 μL of cells (10⁵ cells) transferred to a test tube. 10 μL of PE-conjugated anti-human claudin-4 antibodies (IgG_{2A}) was then added to the test tube and incubated for 30 min. As a control for analysis, cells in a separate tube were treated with a PE-labeled mouse IgG_{2A} isotype control.

### Image Analysis

Immunofluorescence images were acquired and analyzed using Nikon NIS-Elements AR 3.1 software. The software was used to automatically select the cell boundaries and to generate the pixel statistics of the cellular region. The average fluorescence intensity per μm² within the cellular region was determined quantitatively, which allows us to make quantitative comparisons between different cell lines and different antibodies (i.e. different molecular biomarkers). Control experiments included: (1) PEGylated neutral-charge (zwitterionic) QD-L-PEG (no antibody) incubated with pancreatic cancer cell lines and a normal pancreas epithelial cell line (HPDE), and (2) QD-Ab conjugates incubated with HPDE cells.

### Results

### Lipid Encapsulation

The hydrophobic capping ligands on the QDs after synthesis drive the formation of a lipid monolayer, analogous to the outer leaflet in a bilayer membrane. Due to the high curvature of the QDs, a combination of single and double acyl chain phospholipids was used to form the outer leaflet. To determine the optimum composition, QDs were incubated in solution containing different concentrations of a single alkyl chain phospholipid 1-myristoyl-2-hydroxy-sn-glycero-3-phosphocholine (MHPC) and a double alkyl chain lipid 1,2-dipalmitoyl-sn-glycero-3-phosphoethanolamine (DPPE). The yield of the functionalization process was higher than 60% for compositions in the range from 20 to 50 mol % DPPE. For ≦20 mol % DPPE, the QD-L conjugates are monodisperse with an average hydrodynamic diameter of about 13 nm, as expected for the addition of a 2 nm lipid to the 8 nm diameter CdSe/(Cd,Zn)S QDs. In contrast, for ≧30 mol % DPPE, the QDs were polydisperse. The stability in water is also dependent on the lipid composition: QDs with 80 mol % MHPC and 20 mol % DPPE are stable for at least 100 h, significantly longer than other compositions. Replacing the DPPE with a pegylated version (DPPE-PEG2k), resulted in QD-L-PEG conjugates that were stable for several weeks. Finally, the quantum yield of QD-L conjugates was greater than 40% for QDs with 80 mol % MHPC/20 mol % DPPE, and was and significantly higher than other lipid compositions.

### Charge and Antibody-Conjugation

Targeting antibodies were covalently conjugated to the lipid-coated QDs by incorporating a COOH-terminated pegylated lipid (DPPE-PEG2k-COOH). The introduction of charged groups increases stability: QDs that are near-neutral tend to aggregate, resulting in a very low yield after filtration. Conversely, QDs with significant charge exhibit high levels of non-specific cell surface binding in control experiments. Consequently, there is an optimal range of charge (corresponding to a zeta potential of about −10 mV) to minimize aggregation, maximize yield and stability in water, and minimize non-specific binding. Using zwitterionic lipids, the QDs are almost electrically neutral, with a zeta potential of less than 2 mV (FIG. 1c). Introduction of 5 mol % of the COOH-PEG-lipid does not influence the hydrodynamic diameter (FIG. 1b) but results in a small negative surface charge, corresponding to a zeta potential of about −7 mV (FIG. 1c). The antibodies were covalently conjugated to the QDs through formation of an amide bond between the carboxylic acid of the pegylated lipids and primary amines (lysine or N-terminus) on the antibodies. In control experiments, we separated the antibody fragments not covalently linked to the QDs and determined that at least one antibody per QD was active.

Antibody conjugation resulted in an increase in the average hydrodynamic diameter of the QDs from 13 nm to about 21 nm (FIG. 1b) (for a-PSCA) and a small increase in the magnitude of the zeta potential due to the contribution from the antibodies (FIG. 1c). The sharp size distribution and absence of aggregates (FIG. 1b) is characteristic of successful conjugation and is crucial to minimizing non-specific binding for quantitative profiling. The low concentration of carboxylated PEG-lipids minimizes aggregation during antibody-conjugation and charge-induced non-specific binding. The absorbance/emission spectra (FIG. 1d) and the quantum yield (FIG. 1e) of the QDs were not influenced by conjugation and the quantum yield remained more than 40%. With careful removal of excess reagents and filtration, the QDs are stable in water for at least several weeks showing no change in optical properties.

### Profiling

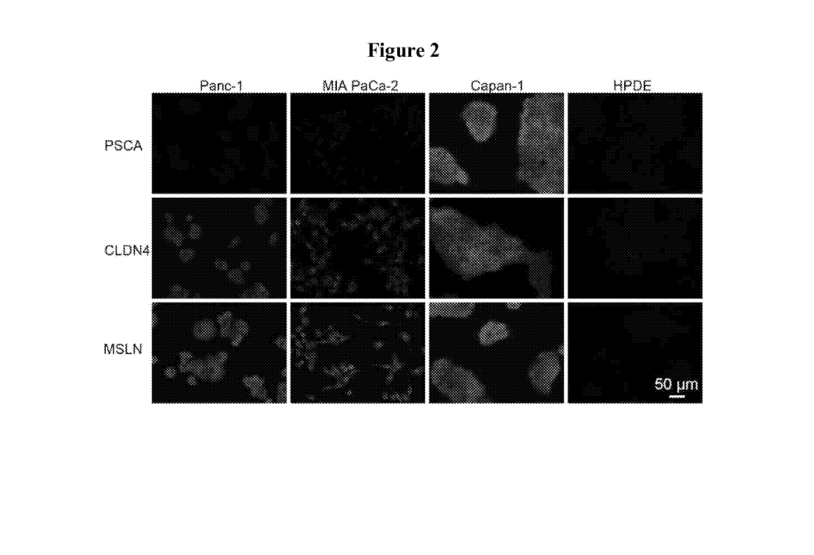

FIG. 2 shows a panel of fluorescence images after incubating Panc-1, MIA PaCa-2, and Capan-1 cells with QD-Ab conjugates. The absence or very low level of fluorescence for HPDE cells or cells incubated with QDs without antibodies indicates that the QD-Ab conjugates exhibit very low non-specific binding. We therefore hypothesize that the fluorescence from the pancreatic cancer cell lines is due to the binding of one QD-Ab conjugate to one target biomarker on the cell surface. This hypothesis is verified in subsequent experiments.

The fluorescence images from the Panc-1 and MIA PaCa-2 cells are very uniform, in part due to the fact that the cells are relatively isolated. In contrast, the fluorescence from the Capan-1 cells is more pronounced at the cell-cell boundaries. The spatial distribution is discussed in more detail below. Qualitative comparison of the fluorescence images in FIG. 2 shows different intensity levels, implying different expression levels. For example, while PSCA shows high expression in Capan-1, MSLN was highly expressed in all three pancreatic cancer cell lines. Similarly, CLDN4 is very highly expressed in Capan-1, moderately expressed in Panc-1, and weekly in expressed MIA PaCA-2. These semi-quantitative observations are in good agreement with results from PCR, Northern blot, and Western blot reported in the literature. (Wente, M. N.; Jain, A.; Kono, E.; Berberat, P. O.; Giese, T.; Reber, H. A.; Friess, H.; Buchler, M. W.; Reiter, R. E.; Hines, O. J., Prostate stem cell antigen is a putative target for immunotherapy in pancreatic cancer. Pancreas 2005, 31, (2), 119-125.) (Michl, P.; Buchholz, M.; Rolke, M.; Kunsch, S.; Lohr, M.; McClane, B.; Tsukita, S.; Leder, G.; Adler, G.; Gress, T. M., Claudin-4: A new target for pancreatic cancer treatment using Clostridium perfringens enterotoxin. Gastroenterology 2001, 121, (3), 678-684.) (Li, M.; Bharadwaj, U.; Zhang, R. X.; Zhang, S.; Mu, H.; Fisher, W. E.; Brunicardi, F. C.; Chen, C. Y.; Yao, Q. Z., Mesothelin is a malignant factor and therapeutic vaccine target for pancreatic cancer. Molecular Cancer Therapeutics 2008, 7, (2), 286-296.) (Argani, P.; Iacobuzio-Donahue, C.; Ryu, B.; Rosty, C.; Goggins, M.; Wilentz, R. E.; Murugesan, S. R.; Leach, S. D.; Jaffee, E.; Yeo, C. J.; Cameron, J. L.; Kern, S. E.; Hruban, R. H., Mesothelin is overexpressed in the vast majority of ductal adenocarcinomas of the pancreas: Identification of a new pancreatic cancer marker by serial analysis of gene expression (SAGE). Clinical Cancer Research 2001, 7, (12), 3862-3868.) We note that these results are only achieved with careful synthesis of the QD-Ab conjugates. Without appropriate functionalization and surface modification, targeting is extremely heterogeneous on the cell surface and control experiments with QDs with no antibody show significant non-specific binding.

To quantitatively determine the expression levels we must (1) confirm that we have saturated all targeted biomarkers on the cell surface and (2) relate the fluorescence intensity to the QD concentration. To confirm that we have saturated all biomarkers on the cell surface, we incubated Panc-1 cells with different concentrations of QD-L-aMSLN conjugates and measured the average fluorescence intensity per cell (FIG. 3a). The fluorescence intensity increases linearly with QD concentration up to 10 pmol, at which point the fluorescence intensity remains constant, indicating that all biomarkers are saturated. Prior to saturation, the slope is 1.0 confirming negligible non-specific binding and no competition for binding sites. Finally, we can conclude that for any QD-Ab/cell line combination, all biomarkers are saturated as long as the fluorescence intensity is ≦240 μm⁻², and this condition is satisfied for all biomarkers and cell lines shown in FIG. 2.

Having established that we have saturated the biomarkers on the cell surface, we next relate the fluorescence intensity to the QD concentration. To quantitatively determine biomarker concentrations over a wide range requires that we vary the exposure time when capturing the fluorescence images. To do this we must consider the time dependence of the emission. FIG. 3b shows results for experiments where Panc-1 cells were incubated with QD-L-aCLDN4 conjugates or claudin-4 antibody conjugated with the fluorophore phycoerythrin (PE, emission 605 nm), PE-aCLDN4. The emission from QD-L-aCLDN4 is constant for at least 10⁴ s while the emission from the PE-aCLDN4 conjugates decreases exponentially with time due to photobleaching. The stable emission for the QDs shows that we can linearly scale fluorescence intensities from different exposure times. Photobleaching results in an exponential decrease in emission for the PE-aCLDN4 conjugates and highlights the difficulty in using fluorophores for quantitative analysis. (Resch-Genger, U.; Grabolle, M.; Cavaliere-Jaricot, S.; Nitschke, R.; Nann, T., Quantum dots versus organic dyes as fluorescent labels. Nature Methods 2008, 5, (9), 763-775.)

To relate the fluorescence intensity to QD concentration, a fixed volume of QD suspension was located between two glass slides (FIG. 3c). By confining the area of the suspension between the glass slides we can relate the fluorescence intensity to an areal density of QDs (FIG. 3d). The average fluorescence intensity per unit area is linearly dependent on the QD concentration and the slope of 1.0 confirms that there are no errors in our procedure.

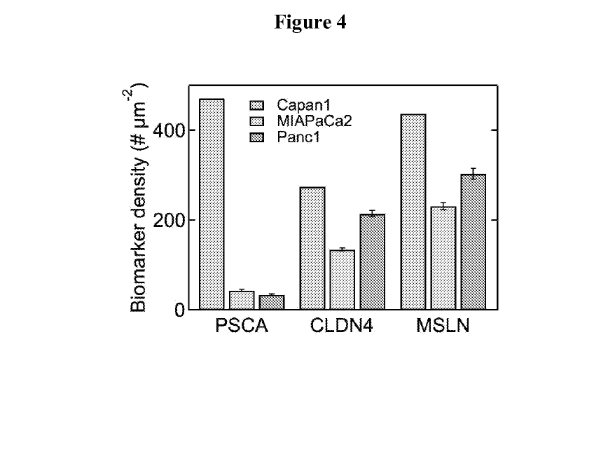

Having established that we have saturated all biomarkers on the cells and that the fluorescence intensity is proportional to the QD concentration, we can quantitatively analyze the fluorescence images. FIG. 4 shows the average biomarker density for PSCA, claudin-4 and mesothelin in the three pancreatic cancer cell lines. The expression levels of these markers are in the range from about 30 μm⁻² to 470 μm⁻². The expression levels for CLDN4 and MSLN on HPDE cells were less than 15 μm⁻² while the expression level for PSCA was about ±4 μm⁻². From analysis of the background intensity we determined a detection limit of about ±4 μm⁻² (SD). The emission from cells incubated with QDs without targeting antibodies corresponds to an average level of non-specific binding of 15 μm⁻², just above the detection limit.

The expression levels of biomarkers can vary depending on passage and genetic drift. Therefore, to validate the biomarker densities we performed flow cytometer analysis for CLDN4 expression on MIA PaCa-2 cells with phycoerythrin (PE)-conjugated anti-CLDN4, allowing us to make a direct comparison to results from QD-Ab conjugates. From control experiments with beads conjugated with known concentrations of PE and the known ratio of PE to antibodies, the number of PE molecules per cell was converted to antibodies per cell. From flow cytometry analysis we obtain an average CLDN4 density on MIA PaCa-2 cells of 121±0.15 μm⁻² (SE, N=5000 cells), in excellent agreement with the value of 135±3.6 μm⁻² obtained from QD-aCLDN4 conjugates (average expression level per cell, N=100 cells).

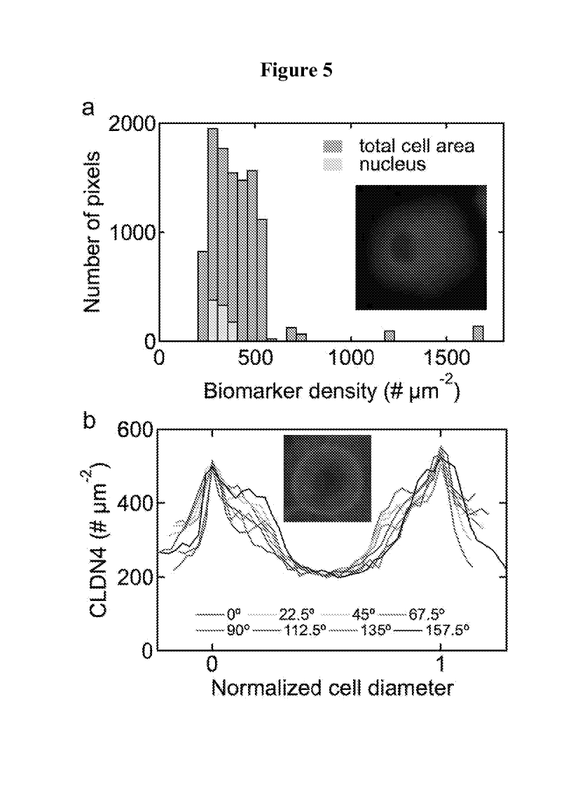

An advantage of biomarker profiling with QD-Ab conjugates, compared to conventional methods such as flow cytometry, is that we can obtain quantitative spatial information at the single cell level. Comparison to the control experiments where cells were incubated with QDs without antibodies, combined with our validation experiments implies that the fluorescence represents the spatial distribution over the cell surface. From the images in FIG. 2, it is evident that the distribution of biomarkers over isolated Panc-1 and MIA PaCa-2 cells is relatively uniform over the cell surface. The nucleus appears somewhat darker since the images were obtained using an inverted microscope. FIG. 5a shows the distribution of mesothelin over a Panc-1 cell. The distribution over the single cell is relatively narrow, 304±0.5 μm⁻² (SE, N=10,802 pixels) indicating relatively uniform expression as inferred from the fluorescence image (inset). The intensity over the nucleus is 288±1.4 μm⁻² (SE, N=912 pixels) only slightly lower than the global cell average (see FIG. 5a). These results also demonstrate that QD aggregation and non-specific binding can be overcome with careful synthesis and design.

In contrast to the Panc-1 and MIA PaCa-2 cells, the Capan-1 cells tend to grow in clusters. The distribution of claudin-4 on capan-1 cells is highly non-uniform with significantly higher intensity at the paracellular junctions, consistent with previous immunofluorescence studies (Michl, P.; Buchholz, M.; Rolke, M.; Kunsch, S.; Lohr, M.; McClane, B.; Tsukita, S.; Leder, G.; Adler, G.; Gress, T. M., Claudin-4: A new target for pancreatic cancer treatment using Clostridium perfringens enterotoxin. Gastroenterology 2001, 121, (3), 678-684.). This paracellular enhancement in cell clusters is expected since claudin-4 is a tight junction protein (Hewitt, K. J.; Agarwal, R.; Morin, P. J., The claudin gene family: expression in normal and neoplastic tissues. BMC Cancer 2006, 6, (186), 1-8.) (Hewitt, K. J.; Agarwal, R.; Morin, P. J., The claudin gene family: expression in normal and neoplastic tissues. BMC Cancer 2006, 6, (186), 1-8.). FIG. 5b shows quantitative linear profiling of the claudin-4 density along a set of eight radial lines through the center of the cell and separated by an angle of 22.5°. In the paracellular regions, the claudin-4 density is around 500 μm⁻², more than double the value in the central region. These results highlight the feasibility of quantitative spatial mapping for isolated cells and monolayer clusters.

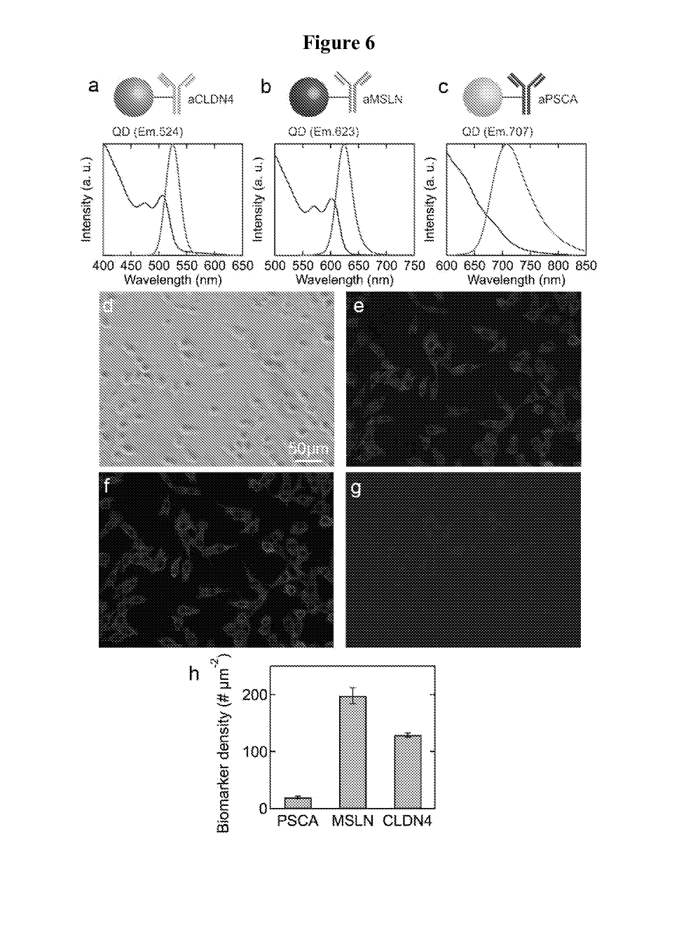

So far we have demonstrated quantitative profiling at the single cell level and spatial profiling. For high throughput profiling of multiple biomarkers, it would be desirable to perform multiplexed imaging. By attaching different antibodies to QDs with different emission wavelength, we prepared color-coded QD-Ab conjugates (see FIG. 6) to demonstrate multiplexed targeting in human pancreatic cancer cell lines: QD(Em.524 nm)-L-aCLDN4 (green), QD(Em.623 nm)-L-aMSLN (red), and QD(Em.707 nm)-L-aPSCA (NIR). The wavelength of each QD was tuned to minimize the overlap of the emission with those of other QDs, but still to be detectable using different emission filters. Equal amounts of the three different color-coded QDs were simultaneously incubated with MIA PaCa-2 cells and FIG. 6 shows the resulting phase contrast image and fluorescence images at the same location taken with different emission filters. Biomarker densities determined from quantitative analysis of the fluorescence images (FIG. 6), are in a good agreement with the results from the individual QD-Ab conjugates (FIG. 2) and analysis (FIG. 6).

### Discussion

We have demonstrated quantitative profiling of biomarkers for pancreatic cancer at the single cell level using QD-Ab conjugates. The key requirements for quantitative profiling of membrane biomarkers using a QD probe are that one QD-Ab conjugate is bound to one target molecule, with no aggregation or non-specific binding. Using our lipid coating strategy for water solubilization and antibody coupling using pegylated lipids, non-specific binding and aggregation are negligible, allowing quantitative profiling of biomarkers for pancreatic cancer.

The expression levels for PSCA, CLDN4, and MSLN in Capan-1, MIA PaCa-2, and Panc-1 cells are in the range from about 30 μm⁻² to 470 μm⁻². The results are in agreement with results from western blot, northern blot, and PCR where expression levels are scored on a relative scale. (Wente, M. N.; Jain, A.; Kono, E.; Berberat, P. O.; Giese, T.; Reber, H. A.; Friess, H.; Buchler, M. W.; Reiter, R. E.; Hines, O. J., Prostate stem cell antigen is a putative target for immunotherapy in pancreatic cancer. Pancreas 2005, 31, (2), 119-125.) (Michl, P.; Buchholz, M.; Rolke, M.; Kunsch, S.; Lohr, M.; McClane, B.; Tsukita, S.; Leder, G.; Adler, G.; Gress, T. M., Claudin-4: A new target for pancreatic cancer treatment using Clostridium perfringens enterotoxin. Gastroenterology 2001, 121, (3), 678-684.) (Li, M.; Bharadwaj, U.; Zhang, R. X.; Zhang, S.; Mu, H.; Fisher, W. E.; Brunicardi, F. C.; Chen, C. Y.; Yao, Q. Z., Mesothelin is a malignant factor and therapeutic vaccine target for pancreatic cancer. Molecular Cancer Therapeutics 2008, 7, (2), 286-296.) (Argani, P.; Iacobuzio-Donahue, C.; Ryu, B.; Rosty, C.; Goggins, M.; Wilentz, R. E.; Murugesan, S. R.; Leach, S. D.; Jaffee, E.; Yeo, C. J.; Cameron, J. L.; Kern, S. E.; Hruban, R. H., Mesothelin is overexpressed in the vast majority of ductal adenocarcinomas of the pancreas: Identification of a new pancreatic cancer marker by serial analysis of gene expression (SAGE). Clinical Cancer Research 2001, 7, (12), 3862-3868.) The highest expression levels were obtained from PSCA and MSLN in Capan-1 cells, and the lowest expression levels were for PSCA in MIA PaCa-2 and Panc-1 cells. Expression levels were validated using flow cytometry to determine the average expression levels for CLDN4 on MIA PaCa-2 cells. The determination of quantitative expression levels allows direct comparison between cell types at the single cell level. Furthermore, we can provide quantitative spatial information on the distribution of biomarkers.

Despite the complexity of these experiments, measurements performed with QDs that were synthesized and functionalized at different times were reproducible. For example, here we report an average expression level for CLDN4 on Panc-1 of 214 μm⁻² (FIG. 4). In independent experiments we measured average expression levels of 228 μm⁻² and 259 μm⁻². Similarly, we measured values for MSLN expression on Panc-1 of 304 μm⁻² (FIG. 6) and 300 μm⁻², and expression levels for PSCA on Panc-1 of 32 μm⁻² (FIG. 4) and 33 μm⁻².

The measured expression levels of 30 μm⁻² to 470 μm⁻² correspond to average biomarker spacings on the cell membrane of 46-190 nm. For a 20 nm diameter QD-Ab conjugate, the maximum expression level that can be measured is 2500 μm⁻². As described above, the detection limit reported here was about ±4 μm⁻² corresponding to an average spacing of 500 nm. Based on the upper limit due to the size of the QD-Ab conjugates and the detection limit, the dynamic range for measurement is almost three orders of magnitude. An important advantage of QDs for profiling is that photobleaching is negligible (FIG. 3b) and hence the intensity is linearly related to exposure time. As a result, longer exposure times can be used when the expression level is low.

We have also demonstrated quantitative multiplexed imaging using color-coded QDs. The expression levels obtained from multiplexed profiling of PSCA, CLDN4, and MSLN in MIA PaCa-2 cells very in excellent agreement with expression levels obtained from single QD-Ab experiments. These results show the feasibility of this technology for staging and forecasting since PSCA, CLDN4, and MSLN are expressed in different stages of progression of pancreatic cancer.

The ability to measure quantitative expression levels of membrane proteins has potential impact in a number of fields. For example, profiling of biomarkers in tissue samples would complement conventional histological staining and morphometric analysis, and may improve staging of disease progression. Similarly, profiling of single cells from blood samples, for example circulating tumor cells, may allow improved diagnosis and clinical management.

### Example 2

### CuInₓSe_{y}/ZnS QDs for Biomedical Imaging

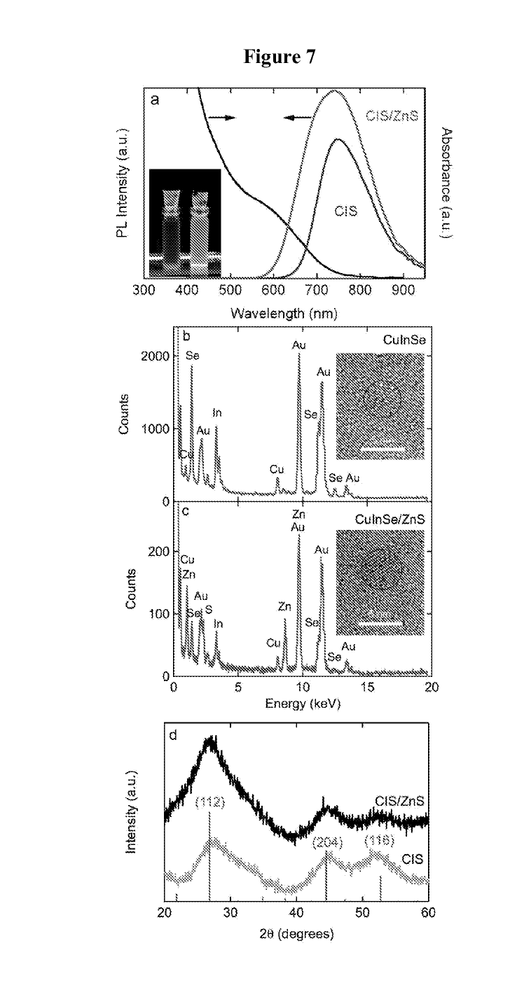

For most applications of QDs, the addition of a wide band gap shell is required to passivate surface states and increase the quantum yield. As we show below, the CuInₓSe_{y} cores have limited stability and hence the shell also serves to isolate the core from the environment. The emission peak at about 745 nm (FIG. 7a) implies a band gap of about 1.66 eV. This is significantly larger than the band gap of 1.26 eV for CuIn₃Se₅ and implies significant confinement. (S. B. Zhang, S. H. Wei, A. Zunger, H. Katayama-Yoshida, Phys Rev B 1998, 57, 9642.) We selected ZnS as a passivation layer since it has a bulk band gap of about 3.68 eV, and is commonly used to passivate II-VI QDs. In addition, the selection of ZnS allows us to avoid possible toxicity concerns by avoiding elements such as cadmium and arsenic. ZnS passivation of CuInₓSe_{y} QDs has been achieved after washing and resuspending the CuInₓSe_{y} cores in ODE/OA prior to introducing the shell precursors and other reagents. (E. Cassette, T. Pons, C. Bouet, M. Helle, L. Bezdetnaya, F. Marchal, B. Dubertret, Chem Mater 2010, 22, 6117.) Here we demonstrate successful passivation after injecting (TMS)₂S and diethyl zinc directly into the suspension of CuInₓSe_{y} cores.

After the growth of the shell, the emission peak is slightly blue-shifted to 737 nm indicating a small decrease in the size of the core due to the formation of an alloy at the core-shell interface (FIG. 7a). The FWHM is increased to 175 nm indicating broader size distribution resulting from the passivation process. (N. S. Pesika, K. J. Stebe, P. C. Searson, J Phys Chem B 2003, 107, 10412.) (N. S. Pesika, K. J. Stebe, P. C. Searson, Adv Mater 2003, 15, 1289.) (J. Park, K. H. Lee, J. F. Galloway, P. C. Searson, J Phys Chem C 2008, 112, 17849.) The core/shell synthesis produced an average emission peak of 741±12 nm with a FWHM of 175±9 nm for 4 syntheses. The quantum yield for the CuInₓSe_{y}/ZnS QDs typically increased to 40-60%, confirming the importance of the passivation of surface states.

FIG. 7c shows a representative EDS spectrum for a CuInₓSe_{y}/ZnS QDs along with a high resolution TEM image (see also Supplemental Information). The EDS spectrum (FIG. 7c) confirms the presence of Zn and S in the CuInₓSe_{y}/ZnS QDs. The average diameter of the core/shell QDs was 5.0±0.2 nm (n=72). The difference in average diameter between the cores and the core/shell QDs implies an average QD shell thickness of about 0.5 nm, in agreement with the expected value based on the concentration of precursors. XRD powder diffraction spectra (FIG. 7d) for the cores and core/shell QDs are consistent with the stannite crystal structure (space group I 4̅2m) for CuIn₃Se₅. (W. Paszkowicz, R. Lewandowska, R. Bacewicz, J Alloy Compd 2004, 362, 241.)

The stability of the QDs was characterized by measuring the time dependence of the quantum yield and PL. The quantum yield of the CuInₓSe_{y} cores in chloroform decreased rapidly after 1-2 days, indicating poor stability. Similar results were obtained for cores synthesized using the method reported by Allen et al. (P. M. Allen, M. G. Bawendi, Journal of the American Chemical Society 2008, 130, 9240.) The loss of stability was largely due to aggregation, as inferred from the fact that the emission peak remained constant at about 760 nm and the FWHM at about 130 nm.

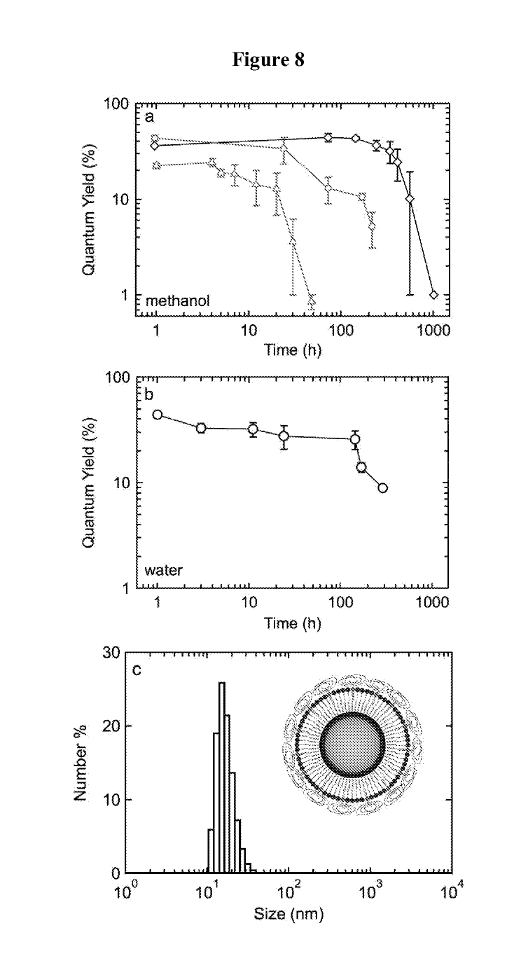

The addition of the ZnS passivation layer resulted in an improvement in stability. The quantum yield in chloroform remained in the range 40-60% for 1-2 days but decreased to 10% after 4-5 days. The PL peak remained constant at about 730 nm and the FWHM remained at about 170 nm. Significant improvements in stability were obtained by replacing the TOPO/HDA coordinating ligands by dodecanethiol (DDT). The quantum yield for DDT-modified CuInₓSe_{y}/ZnS/DDT QDs in chloroform remained high for 10-14 days, and decreased to 10% after 21 days (FIG. 8a).

Lipid coating was used to transfer the CuInₓSe_{y}/ZnS QDs to water. (B. Dubertret, P. Skourides, D. J. Norris, V. Noireaux, A. H. Brivanlou, A. Libchaber, Science 2002, 298, 1759.) Various combinations of single acyl chain lipid and double acyl chain lipids with PEG groups were tested. A lipid composition of 80% PEGylated lipid with 20% single acyl chain lipid gave the best results. These lipid coated QDs showed a quantum yield of about 50% QY in water and were stable for at least several days at room temperature (FIG. 8b). After lipid coating, the average hydrodynamic diameter, measured by DLS, was 15 nm (FIG. 8c). As described above, the core/shell QDs are about 5 nm in diameter. Taking the DDT inner leaflet as 1 nm, the lipid outer leaflet as 2 nm, and the PEG radius of gyration as 2 nm, we expect the overall size to be about 15 nm, in excellent agreement with the measured particle size.

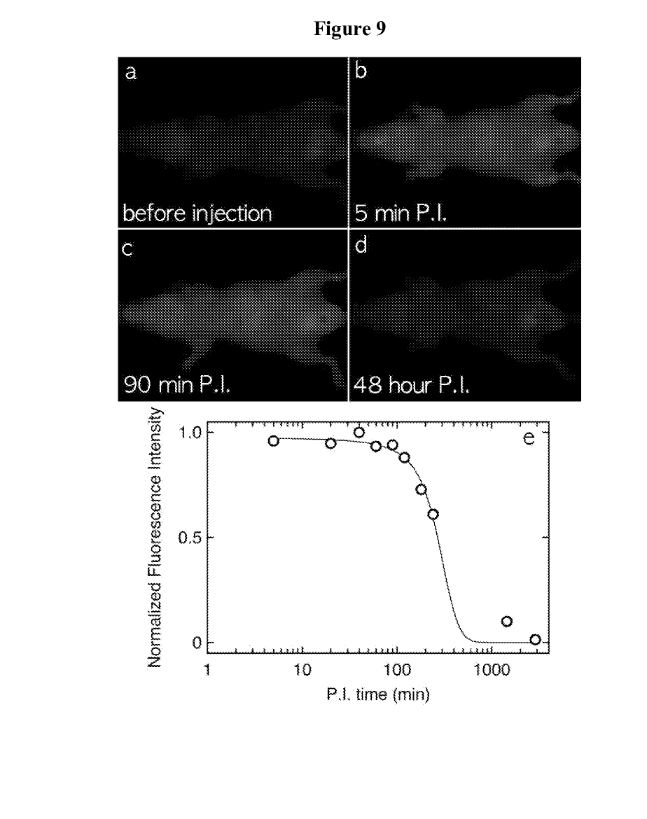

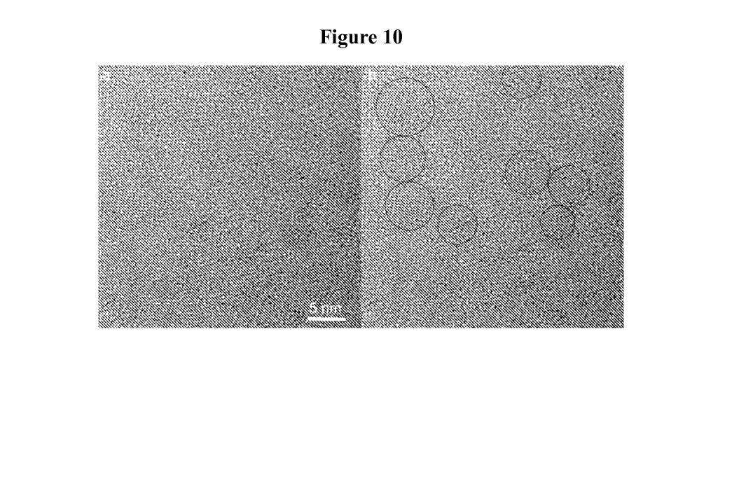

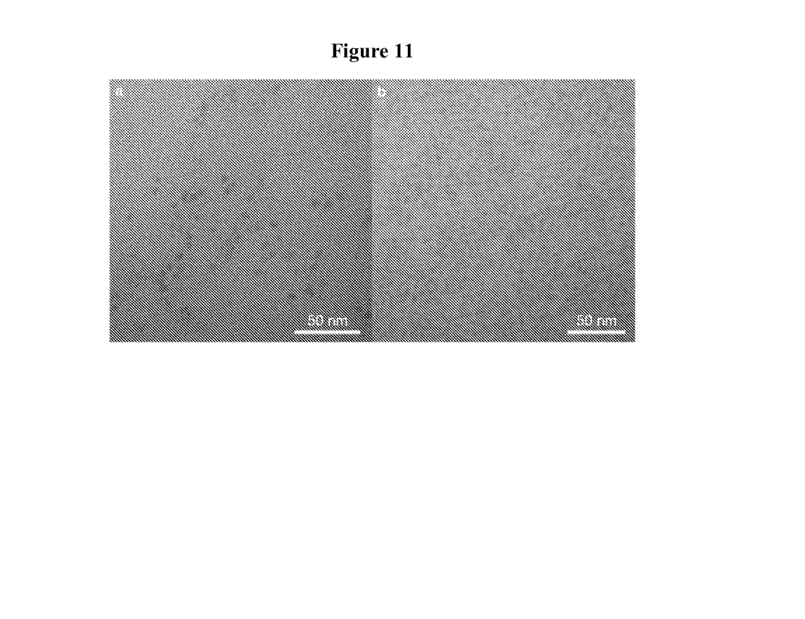

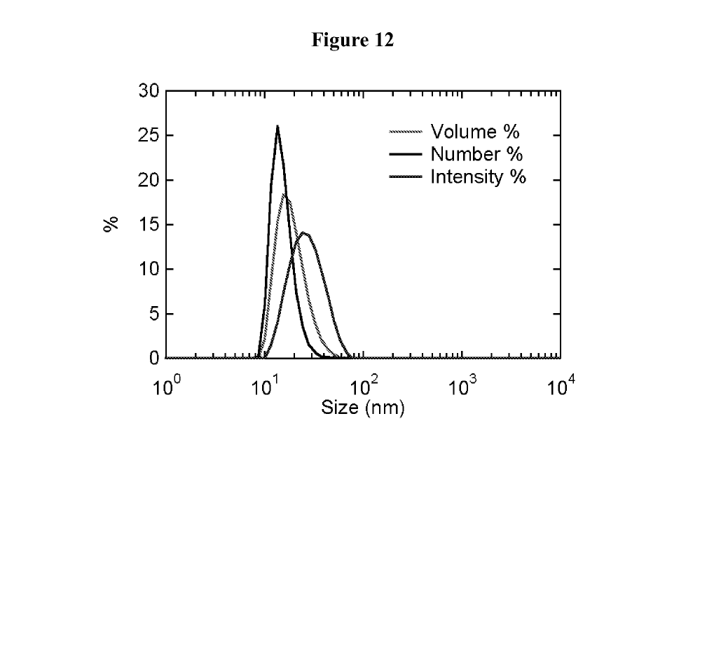

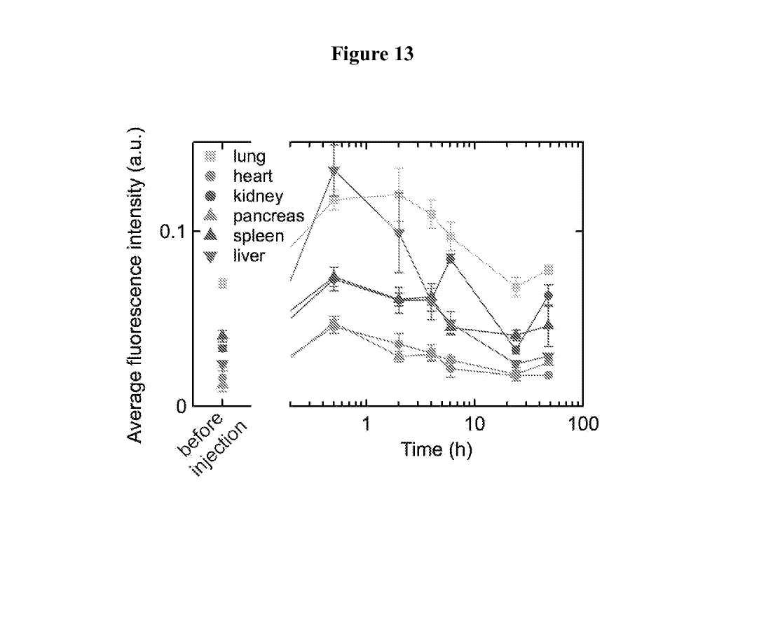

To explore the performance of the CuInₓSe_{y}/ZnS QDs for biomedical imaging, we performed fluorescence imaging in mice after tail vein injection. 230 pmol of lipid coated QDs in 120 ul of saline were introduced by tail-vain injection. Fluorescence images were taken as a function of time post-injection (p.i.). FIG. 9 shows fluorescence images recorded before injection, and at 5 minutes, 90 minutes, and 48 h p.i. Immediately after injection (FIG. 9b) the fluorescence intensity increased relatively uniformly over the whole body of the mouse. Indeed, some of the larger blood vessels were easily detectable. The fluorescence intensity started to decay at 90 minutes p.i. (FIG. 9c), and after 48 h had returned to the same level as before injection (FIG. 9d). Very similar results were obtained for the other mice. There are no bright spots indicating aggregation or measurable accumulation in organs such as the liver or spleen, suggesting good clearance from the body although this remains to be confirmed by quantitative analysis. Fluorescence images of the resected organs (see Supplemental Information) show a similar dependence on time as the dorsal and ventral images; the fluorescence increases to a maximum at about 30 minutes p.i., but then decreases to values close to background after 24 h.

The kinetics of circulation were analyzed quantitatively by determining the average intensity per pixel over the whole image (FIG. 9e). The average intensity remains constant for about 2 hours p.i. and then decreases to the background level before injection after 48 hours. By fitting the data to the function:

$$\frac{I ( t ) - {I}_{background}}{{I}_{max} - {I}_{background}} = \frac{1}{1 + exp ( \frac{t - {t}_{1 \text{/} 2}}{τ} )}$$

we obtain an average retention time t_{1/2} of 268±16 mins and a clearance time τ of 74.5±11.9 mins.

These QDs are externally similar to the high density lipoprotein (HDL) particles in the body that carry cholesterol to the liver for clearance. HDL particles are lipid coated particles with diameter in the range from 10-15 nm and circulate freely in the body. As a result of these unique properties, modified natural HDL particles and biomimetic HDL particles have been explored as contrast agents for MRI. (D. P. Cormode, P. A. Jarzyna, W. J. M. Mulder, Z. A. Fayad, Adv Drug Deliver Rev 2010, 62, 329.) Thus we hypothesize that the good circulation characteristics are due to the size and lipid coating.

In summary, we have demonstrated a one-pot synthesis of CuInₓSe_{y}/ZnS QDs with emission in the near IR, high quantum yield, and good stability. The synthesis is relatively straightforward and reproducible, and avoids the use of elements such as Cd and As. We also have demonstrated that lipid coated CuInₓSe_{y}/ZnS QDs are good candidates for in vivo imaging.

### EXPERIMENTAL

Cu/In Precursor Solution:

Copper iodide (0.045 mmol, CuI, Alfa Aesar, puratonic, 99.999%) and indium iodide (0.18 mmol, InI₃, Alfa Aesar, anhydrous, 99.999%) were mixed with trioctylphosphine (3 ml TOP, Strem, 97%) in a glove box. The solution was stirred at 90° C. for several hours. The precursor solution was stored in the dark and was stable for up to two weeks.

Core Synthesis:

Trioctylphosphine oxide (3.6 g, TOPO, Sigma Aldrich, tech. grade, 90%) and hexadecylamine (6 g, HDA, Sigma Aldrich, tech. grade, 90%) were added to a 100 ml 3-neck flask and heated to 100° C. in vacuum to form a transparent solution. The Cu/In precursor solution was injected into the reaction flask and vacuumed for at least two hours. A more concentrated precursor solution can be used (3 times more concentrated) in order to decrease the amount of TOP. Reducing the amount of TOP makes the washing steps somewhat easier. The syringe and the flasks were wrapped with aluminum foil in order to minimize exposure to light. Next, the temperature was increased to 270° C. in Ar (Airgas, ultra high purity, grade 5) flow. Bis(trimethylsilyl) selenide (150 μl, (TMS)₂Se, Gelest) in TOP (0.5 ml) were mixed in a glove box and injected into the reaction flask. After 6 seconds, 4 ml hexane was injected to quench the reaction. The reaction mixture was then left to cool to 130° C. While injecting hexane into the hot solution, a needle was placed in the septum to avoid a rapid increase in pressure in the flask.

ZnS Coating:

Bis(trimethylsilyl)sulfide (227 μl, (TMS)₂S, Sigma Aldrich, synthesis grade) and diethyl zinc (115 μl, Sigma Aldrich, 52.0 wt. % Zn) were mixed with TOP (1 ml) in a glove box and injected into the suspension of CuInₓSe_{y} cores at 130° C. Diethyl zinc is very reactive and should be handled with care. These precursor solutions were placed in a secondary container when transferring from the glove box to the hood to minimize exposure to air. Best results were obtained with fresh chemicals, typically within a month of opening. The amounts of Zn and S were calculated to achieve 3 monolayers (ML) ZnS on the CuInₓSe_{y} cores (J. F. Galloway, J. Park, K. H. Lee, D. Wirtz, P. C. Searson, Science of Advanced Materials 2009, 1, 1.). After injecting the precursors for the shell, the reaction mixture was cooled to 85° C. and the QDs annealed for 2 hours. This annealing time was found to give the maximum quantum yield.

Dodecanethiol Functionalization:

After annealing the CuInₓSe_{y}/ZnS QDs for 2 hours, dodecanethiol (1 ml, DDT, Sigma Aldrich, ≧98%) was injected into the QD suspension. Final solutions were poured into two 15 ml centrifugal tubes. Methanol and isopropyl alcohol (8:2 by volume) were added to the tubes until they were full. Using stronger solvents degraded the surface of QDs and resulted in aggregation. Too many washing steps (usually more than 3 times) also resulted in aggregation. The QD suspensions were centrifuged at 8000 rpm for 3 minutes. After centrifugation, the precipitate was re-dispersed in hexane and the same washing steps repeated at least twice. The final precipitate was re-dispersed in chloroform.

Water Solubilization:

DI-water (2 ml) was added to a 5 ml vial. A stirring rod was inserted into the vial to ensure good mixing. This vial was placed in a beaker containing glycerol maintained at a temperature of 110° C. using a hot plate. In a separate vial, polyethylene glycol oleyl ether (0.61 μmol, Brij93®,Sigma Aldrich), 1,2-distearoyl-sn-glycero-3-phosphoethanolamine-N-[methoxy(polyethylene glycol)-2000] (2.43 μmol, DSPE-PEG2k, Avanti Polar Lipids), and 2.3×10¹⁴ QDs were mixed thoroughly. The amount of lipids corresponds to a 20-fold excess of with respect to the amount required for complete coverage of the QDs. This mixture was sonicated and then added drop-wise to the DI-water at 100° C. under vigorous stirring for 2 minutes. The solution was then centrifuged at 4000 rpm for 3 minutes and the supernatant filtered through 200 nm syringe filter.

In Vivo Imaging:

Three mice were prepared for tail vain injection. Special food (TD97184, Teklad Purified Diet, Harlan) was fed a week prior to the experiment in order to eliminate auto-fluorescence from the food. 120 μl QD solution containing 60 μl of QDs and 60 μl of saline was injected into the tail vein and imaged using a Li-cor imaging system in the Small Animal Imaging Facility. The QD concentration was determined from absorbance measurements using an extinction coefficient of 3.1×10⁶ cm² mol⁻¹, based on the number of moles of the solid phase. The extinction coefficient was determined from gravimetric measurements using a density of 3.49 g cm⁻³ for CuIn₃Se₅. Fluorescence images were taken at different time points. Procedures were conducted according to protocols approved by Johns Hopkins Animal Care and Use Committee.

Characterization:

Photoluminescence (PL) measurements were obtained using a fluorometer (Fluorolog-3 fluorometer, Horiba Jobin Yvon). Absorbance spectra were obtained using a spectrophotometer (Cary 50 UV/vis). Suspensions of QDs in chloroform or in water were placed in cuvettes with polished sides (Starna Cells, Inc.). Transmission electron microscope images and EDS data were obtained using a Philips EM 420 TEM and FEI Tecnai 12 TWIN. High resolution images were obtained using a Philips CM 300 FEG TEM. Samples for transmission electron microscopy were prepared by placing a drop of the QD suspension on a gold lacey-carbon grid. The absolute QY was measured using an Absolute PL Quantum Yield Measurement System (Hamamatsu, C9920-02). Particle size distributions were measured using a Malvern Zetasizer. A Pearl Impulse Li-Cor system was used for small animal imaging. Pearl Impulse software and ImageJ were used for analysis of fluorescence images. XRD measurements were performed using a Phillip's X Pert 3040 with a Cu K_{α} source.

### Example 3

QD Synthesis

TABLE 1
Summary of 101 synthesis experiments for CIS and CIS/ZnS QDs.

| Surfactants | Core | Core/shell | |
|---|---|---|---|
| (number of | synthesis | synthesis | |
| experiments) | with QY > 15% | with QY > 30% | Comments |
| OA (36) | 19% | 0% | QY decreased |
| | | | after ZnS |
| | | | passivation (average |
| | | | decrease 3%, max. |
| | | | decrease 13%) |
| TOPO/HDA | 52% | 60% | QY = 32 ± 14 |
| (23) | | | (maximum 50%) |
| one pot with | N/A | 60% | QY = 32 ± 11 |
| TOPO/HDA | | | (maximum 60%) |
| (42) | | | |

The Cu, In, Se, Zn, and S precursors were the same in all experiments. 0.045 mmol CuI and 0.18 mmol InI₃ were mixed in 3 ml of TOP; 150 μl (TMS)₂Se; 115 μl diethyl zinc; 227 μl bis(trimethylsilyl)sulfide in TOP.

1. Olyamine (OA)

Cores: precursors injected into OA (T=260-290° C.; t=15-40 s). Annealing (t=0-1 h) was performed at 100° C. Cores washed with methanol, acetone, ethanol, hexane, or chloroform. Synthesis of ZnS in TOPO/HDA (T=100-240° C.) Annealing (t=0-10 h) was performed at temperatures from 100-240° C. CIS/ZnS QDs were washed with methanol, acetone, ethanol, hexane, or chloroform.

2. Trioctylphosphine Oxide and Hexadecylamine (TOPO/HDA)

Cores: precursors injected into TOPO/HDA (T=250-300° C., t=2-50 s) Annealing (t=0 to 2 h) was performed at 90° C. Cores were washed with methanol, acetone, ethanol, methanol/isopropyl, hexane, or chloroform. Synthesis of ZnS in TOPO/HDA (T=130-220° C.) Annealing (t=0-20 h) was performed at temperatures from 85-220° C. CIS/ZnS QDs were washed with methanol, acetone, ethanol, methanol/isopropyl, hexane, or chloroform.

3. One-Pot TOPO/HDA

Core precursor injected into TOPO/HDA at 270° C. Reaction time from 6-50 s. Synthesis of ZnS in TOPO/HDA (T=70-240° C.) Annealing (t=0-20 hours) was performed at temperatures from 85-220° C. CIS/ZnS QDs were washed with methanol, acetone, ethanol, methanol/isopropyl, hexane, or chloroform.

The one-pot synthesis produces excellent quantum yield and stability, and also reduces the number of washing steps, synthesis time, and cost, compared to the two-step synthesis.

The addition of DDT (up to 1 ml) did not enhance quantum yield, but it significantly improved stability.

What is claimed is:

1. A method of preparing quantum dots synthesized comprising a reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio to produce a CuInₓSe_{y} core, wherein x=1-4 and y=2-6.

2. The method of claim 1, further comprising addition of a ZnS shell to the CuInₓSe_{y} core.

3. The method of claim 2, further comprising thiolation of the CuInₓSe_{y}/ZnS quantum dot.

4. The method of claim 3, further comprising lipid encapsulation of the thiolated CuInₓSe_{y}/ZnS quantum dot.

5. The method of claim 4, further comprising antibody conjugation of the lipid encapsulated, thiolated CuInₓSe_{y}/ZnS quantum dot.

6. A quantum dot comprising a CuInₓSe_{y}/ZnS core/shell synthesized by reaction of CuI, InI₃, and bis(trimethylsilyl) selenide ((TMS))₂Se) in trioctylphosphine oxide (TOPO) and hexadecylamine (HDA) wherein the TOPO/HDA is present in a 1:3 ratio, wherein x=1-4 and y=2-6.

7. The quantum dot of claim 6, further comprising thiolation of the quantum dot.

8. The quantum dot of claim 7, further comprising lipid encapsulation of the quantum dot.

9. The quantum dot of claim 8, further comprising conjugation of the quantum dot to an antibody.

10. A method of in vivo imaging of a subject comprising introduction of the quantum dot of claim 9 into a subject;
imaging the subject; and
recordation of the image.

11. The method of claim 10, wherein introduction of the quantum dot to a subject can be oral or parenteral.

12. The method of claim 10, wherein the imaging of the subject is fluorescence imaging.

13. The method of claim 10, wherein the recordation of the image is by digital image recording.
